(19) United States
(12) Patent Application Publication
(10) Pub. No.: US 2023/0094575 A1
(43) Pub. Date: Mar. 30, 2023

(54) IMPROVED SPINAL IMPLANT

(71) Applicant: Board of Regents, The University of Texas System, Austin, TX (US)

(72) Inventors: Justin Earl BIRD, HOUSTON, TX (US); Nicolas M. CORDARO, Houston, TX (US); Jeffrey M. BRITTAN, Houston, TX (US)

(73) Assignee: Board of Regents, The University of Texas System, Austin, TX (US)

(21) Appl. No.: 17/906,334

(22) PCT Filed: Mar. 15, 2021
(86) PCT No.: PCT/US2021/022432

§ 371 Date: Sep. 14, 2022

(51) Int. Cl. A61F 2/44

(52) U.S. Cl. CPC A61F 2/4455; A61F 2002/30593

(57) ABSTRACT

This disclosure describes spinal implants with anchoring elements including an aperture for delivery of injectable materials. In one aspect, a spinal implant includes a body defining one or more injection ports and one or more channels, the one or more injection ports configured to receive flowable material and to provide the flowable material to the one or more channels; and one or more anchoring elements protruding from a surface of the body, the one or more anchoring elements each defining an aperture coupled to the one or more channels and configured to receive the flowable material from the one or more channels and to provide/output the flowable material from the aperture.

### CROSS-REFERENCE TO RELATED APPLICATIONS

This application claims the benefit of U.S. Provisional Application No. 62/990,079, filed Mar. 16, 2020, which is hereby incorporated by reference in its entirety.

### TECHNICAL FIELD

Aspects of the present disclosure relate generally to spinal implants, and more specifically, but not by way of limitation, to spinal implants for spinal fusion.

### BACKGROUND

Spinal fusion surgeries use implants to fuse together, i.e., connect, two adjacent vertebrae. One main complication in spinal fusion surgery is that fusion may not be achieved or will be delayed in up to twenty percent of all cases. Such complications are often referred to as non-union. Subsidence is another common complication, in which the bone surrounding a spinal implant collapses around the implant. When non-union or subsidence occurs, spine alignment issues may occur and/or recurrent stenosis may occur.

Certain patients are more prone to prone to such nonunion/subsidence issues. For example, patients with osteoporosis, diabetes mellitus (DM), cancer (e.g., multiple myeloma), and/or radiated bone, and patients who smoke.

Current spinal fusion implants are designed to fit between vertebral bodies without disrupting vertebral endplates. This creates technical challenges regarding end plate preparation and fusion.

Current spinal fusion implants are inherently unstable under certain spinal motions and can easily shift with normal spinal motion. For example, the actual bone surrounding the implant may not be supported structurally by the implant. Some such spinal fusion implants require screw fixation and/or overlying plates to attach the implant to the surrounding bones. Such fasteners and additional components increases the complexity of the surgery and enlarges a size of the implant which increases the risk for complications.

### SUMMARY

This disclosure describes spinal implants that can be attached directly to the bone and methods of manufacturing and using such spinal implants. The spinal implants described herein include one or more anchoring elements configured to be inserted into bone (e.g., cancellous bone) of one or more spinal vertebrae and which include channels or ducts to deliver flowable (e.g., injectable material) to the bone. For example, a spinal implant may include keels with apertures (e.g., boreholes) which are coupled to injection tubes such that the spinal implant may receive one or more materials in vivo and provide the materials directly to an interior of a bone via an interior pathway of the spinal implant. By securing the spinal implant directly to the bone, a more secure and more robust fusion can be achieved with lower non-union and subsidence than conventional spinal implants.

In a particular implementation, a spinal implant includes a body defining one or more injection ports and one or more channels, the one or more injection ports configured to receive flowable material and to provide the flowable material to the one or more channels; and one or more anchoring elements protruding from a surface of the body, the one or more anchoring elements each defining an aperture coupled to the one or more channels and configured to receive the flowable material from the one or more channels and to provide/output the flowable material from the aperture.

In another particular implementation, a method of using a spinal implant includes accessing a spinal implant in vivo; and injecting a flowable material into a void (e.g., prepared groove) of a vertebral body via an aperture in an anchoring element and a channel defined by a body of the spinal implant.

In another particular implementation, a method of using a spinal implant includes accessing a vertebral cavity; preparing a vertebral body for the spinal implant by removing a portion of a vertebral endplate and a portion of cancellous bone of the vertebral body to create a void in the vertebral body; inserting an anchoring element of the spinal implant into the void; and driving the anchoring element of the spinal implant into the void to position the spinal implant.

In another particular implementation, a method of using a spinal implant includes receiving, by a channel of an in vivo spinal implant, a flowable material; providing, by the spinal implant, the flowable material to one or more anchoring elements through a body of the spinal implant; and emitting, by an aperture of the one or more anchoring elements, the flowable material to a cancellous bone within a vertebral body.

In another particular implementation, a method of manufacturing a spinal implant includes measuring a patient for a spinal implant to determine one or more measurements; modifying at least one base dimension of the spinal implant based on the one or more measurements; generating model data based on the modified dimension; and three dimensionally printing the spinal implant based on the model data.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically. Additionally, two items that are “coupled” may be unitary with each other. To illustrate, components may be coupled by virtue of physical proximity, being integral to a single structure, or being formed from the same piece of material. Coupling may also include mechanical, thermal, electrical, communicational (e.g., wired or wireless), or chemical coupling (such as a chemical bond) in some contexts.

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. As used herein, the term “approximately” may be substituted with “within 10 percent of” what is specified. Additionally, the term “substantially” may be substituted with “within [a percentage] of” what is specified, where the percentage includes 0.1, 1, or 5 percent; or may be understood to mean with a design, manufacture, or measurement tolerance. The phrase “and/or” means and or. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), and “include” (and any form of include, such as “includes” and “including”). As a result, an apparatus that “comprises,” “has,” or “includes” one or more elements possesses those one or more elements, but is not limited to possessing only those one or more elements. Likewise, a method that “comprises,” “has,” or “includes” one or more steps possesses those one or more steps, but is not limited to possessing only those one or more steps.

Any aspect of any of the systems, methods, and article of manufacture can consist of or consist essentially of—rather than comprise/have/include—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. Additionally, it will be understood that the term “wherein” may be used interchangeably with “where.”

Further, a device or system that is configured in a certain way is configured in at least that way, but it can also be configured in other ways than those specifically described. The feature or features of one embodiment may be applied to other embodiments, even though not described or illustrated, unless expressly prohibited by this disclosure or the nature of the embodiments.

Some details associated with the aspects of the present disclosure are described above, and others are described below. Other implementations, advantages, and features of the present disclosure will become apparent after review of the entire application, including the following sections: Brief Description of the Drawings, Detailed Description, and the Claims.

### BRIEF DESCRIPTION OF THE DRAWINGS

A further understanding of the nature and advantages of the present disclosure may be realized by reference to the following drawings. The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 is a block diagram of an example of a spinal implant;

FIG. 2A is a top view of an example of a spinal implant of FIG. 1;

FIG. 2B is a side view of the example spinal implant of FIG. 1A;

FIG. 2C is a bottom view of the example spinal of FIG. 1A;

FIG. 3 is a perspective view of the example of the spinal implant of FIG. 1A;

FIG. 4 is another perspective view of the example of the spinal implant of FIG. 1A;

FIG. 5A is another side view (profile view) of the example spinal implant of FIG. 1A depicting a section line;

FIG. 5B is a side cross-section view of the example spinal implant taken along the section line of FIG. 5A;

FIG. 6 is a perspective view of another example of a spinal implant including a porous surface;

FIG. 7 is a diagram illustrating a spinal implant inserted into a lumbar portion of a spine;

FIG. 8 is a perspective view of yet another example of a spinal implant;

FIG. 9 is a flowchart illustrating an example of a method of using a spinal implant;

FIG. 10 is a flowchart illustrating an example of a method of using a spinal implant;

FIG. 11 is a flowchart illustrating an example of a method of using a spinal implant;

FIG. 12 is a flowchart illustrating an example of a method of manufacturing a spinal implant;

FIG. 13 is an image illustrating an injector device coupled to an injection port.

FIG. 14 is an image illustrating bone site preparation.

FIG. 15 is an image illustrating spinal implant insertion.

FIG. 16 is an image illustrating spinal implants prior to insertion.

FIG. 17 is an image illustrating in vivo spinal implants.

FIG. 18 is an image illustrating in vivo injection of flowable materials to a spinal implant.

FIG. 19 is an image illustrating spinal implants after flowable material has been injected.

FIG. 20 is a CT image illustrating spinal implants after insertion and injection.

FIG. 21 is an X-ray image illustrating spinal implants after insertion and injection.

FIG. 22 is an image illustrating periosteum and bone resected demonstrating cement in implant and bone.

FIG. 23 is an image illustrating of a transected L3 vertebral body demonstrating PMMA within the L3 vertebral body; and

FIG. 24 is an image illustrating an additional view after implant extraction.

### DETAILED DESCRIPTION

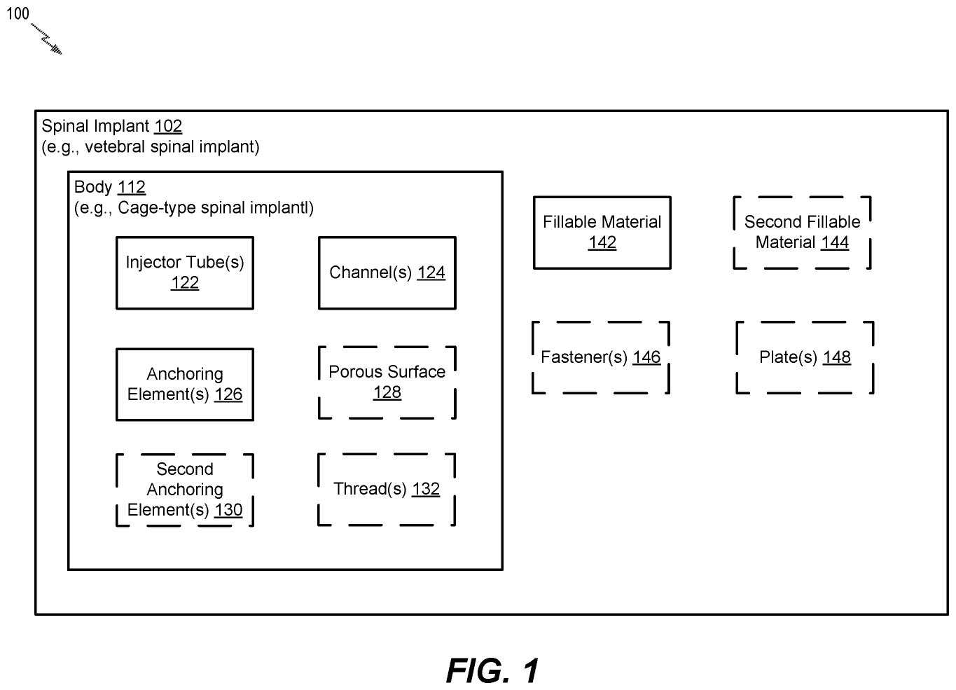

FIG. 1 illustrates a block diagram 100 of a spinal implant 102. Spinal implant 102 includes a body 112 and is configured to receive one or more flowable materials. For example, first fillable material 142, second fillable material 144, or both. Spinal implant 102 may include one or more other components in other implementations. In some implementations, spinal implant 102 includes or corresponds to a vertebral implant or an intervertebral implant, such as a disc type implant or a cage type implant. In other implementations, spinal implant 102 includes or corresponds to another type of spinal implant.

Body 112 includes one or more injector tubes 122, one or more channels 124, and one or more anchoring elements 126. For example, body 112 may define one or more of 122-126. Body 112 may include a one-piece/monolithic structure or may include multiple distinct pieces which make up the body 112. Body 112 may include a single material or may be a composite of multiple materials. For example, body 112 may include a metal, a plastic or a composite/laminate material. To illustrate, body 112 may be titanium, PEEK, or carbon fiber. As another example of composite materials, the body 112 may include two or more materials, such as a PEEK core with a titanium exterior.

The one or more injector tubes 122 are configured to receive flowable material. For example, the one or more injector tubes 122 may be configured to receive injected flowable material (e.g., 142. 144, or both) which may fill up at least a portion of the body 112. The one or more injector tubes 122 are operatively coupled to the one or more channels 124. For example, each injector tubes of the one or more injector tubes 122 may be coupled to a corresponding channel of the one or more channels 124 in some implementations. Additionally, or alternatively, a single injector tube may be coupled to a plurality of channels. The one or more channels 124 are configured to provide the received flowable material to the one or more anchoring elements 126.

The one or more anchoring elements 126 are configured to be inserted into bone and to provide the flowable material to the bone. For example, the one or more anchoring elements 126 protrude from a surface of the body 112 and are configured to be inserted into voids formed in the bone. To illustrate, grooves may be made in vertebral endplates and cancellous bone and the anchoring elements 126 may be inserted into corresponding grooves. The one or more anchoring elements 126 may include one or more delivery elements to provide/output the flowable material. For example, the anchoring elements 126 may be open, include one or more apertures (e.g., boreholes), etc.

The one or more anchoring elements 126 may include or correspond to keels, stems, or a combination thereof. Because of the anchoring elements 126 insertion into the bone and the delivery of flowable material, the anchoring elements 126 may deliver bone cement and/or grafts to the interior of the bone to secure the spinal implant 102 in place.

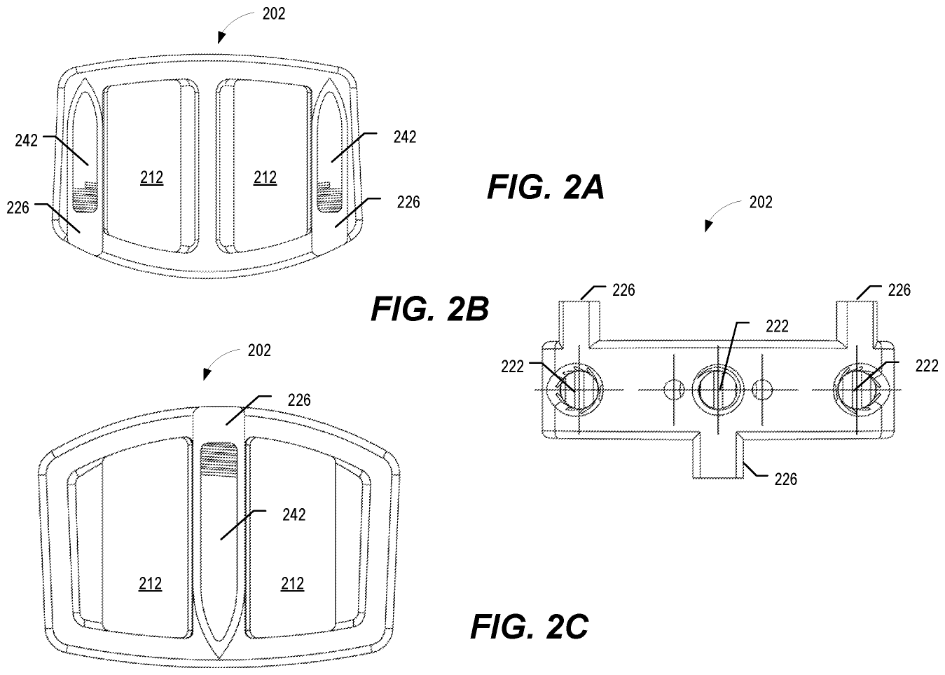

In some implementations, body 112 further includes or defines one or more openings or hollows. For example, the body 112 may define one or more interior openings such that material can pass through a portion of the body 112, as illustrated in at least FIG. 2A. The openings or hollows may promote flexibility and bone ingrowth.

Additionally, or alternatively, the spinal implant 102 includes a porous surface 128. For example, at least a portion of a surface of the body 112 is porous. The porous surface 128 may enable better adhesion for bone cement and promote cellular growth and increase bone graft effectiveness. As an illustrative example, a majority of the surface of the body 112 may be porous titanium. In some example, only a portion of the surface of the body is porous, such as the anchoring elements 126.

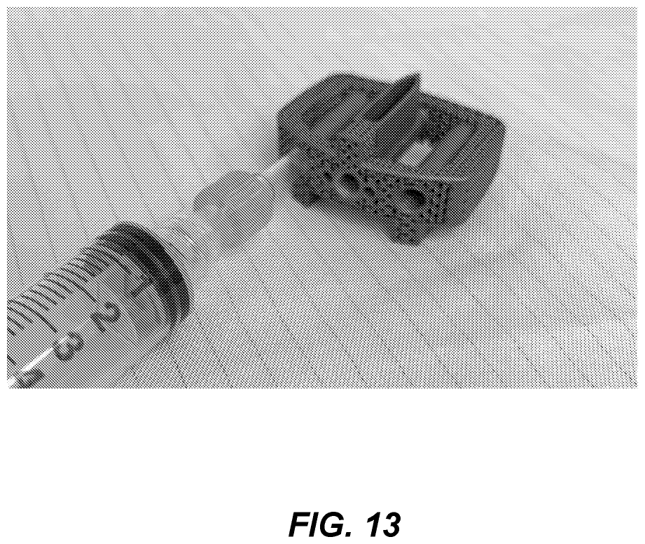

In some implementations, the spinal implant 102 includes one or more threads 132. For example, at least one of the one or more injector tubes 122 may include threads 132. The threads 132 may be configured to mate with corresponding threads of an injector device (shown in FIG. 13). The threads 132 may be configured to form a seal when the injector device is inserted into the body 112, such that injectable material does not leak out.

The spinal implant 102 is configured to receive one or more flowable materials and to provide the flowable materials to vertebral endplates, such as an interior thereof. In a particular implementation, the spinal implant 102 is configured to receive a first fillable material 142. In some implementations, the spinal implant 102 may optionally be configured to receive a second fillable material 144. The first and second fillable materials 142 and 144 may include or correspond to a combination of materials including, but not limited to adhesives (e.g., bone cement), biologic bone void fillers, drugs, other biologics, or a combination thereof. As illustrative examples of the preceding categories bone cement may include PMMA, biologic bone void fillers ma include formulations including hydroxyapatites, calcium sulfates, magnesium, hyaluronic acid, and/or collagens. Additionally, the drugs may include antibiotics (i.e., Gentamycin, tobramycin) and antiresorptives (bisphosphonates, denosumab, etc., and the other biologics may include Bone morphogeneic proteins, stem cells, or a combination thereof. The first and second fillable materials 142 and 144 may be injected together (e.g., mixed), sequentially, or

In some implementations, the spinal implant 102 may be configured to be coupled to one or more fasteners 146. For example, the fasteners 146 may include screws, pins, rods, etc. To illustrate, screw holes defined by body 112 may receive fasteners 146 (e.g., bone screws) to secure the spinal implant 102, either together, to another component, to bone, or a combination thereof.

Additionally, or alternatively, the spinal implant 102 may include one or more plates 148. A plate 148 may include or correspond to a flat piece of material configured to attach to one or more vertebral endplates and the body 112. The plate(s) 148 may provide additional reinforcement or connect multiple spinal implants, such as multiple bodies/cages.

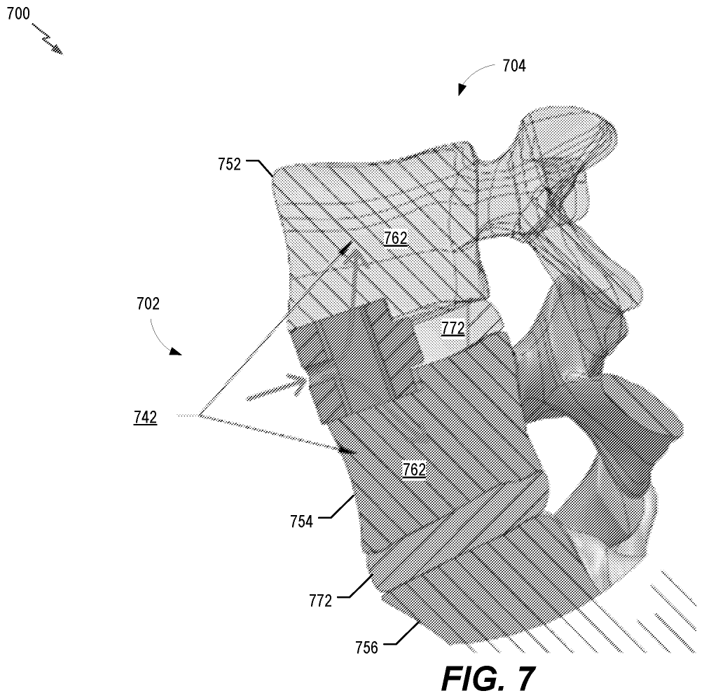

Operation of the spinal implant 102 and other spinal implants is described with reference to FIGS. 7, 9-11, and 13-24. Manufacturing of the spinal implant is described with reference to FIG. 8.

Thus, spinal implant 112 includes anchoring elements 126 configured to deliver flowable material to and into bone. Accordingly, spinal implant 102 has increased adhesion to the bone, as compared to conventional spinal implants that use screws and/or use adhesives external to the spinal implant or deliver the adhesives to a surface of a bone. Consequently, spinal implant 102 may enable better and quicker fusion of the spinal implant and reduce non-union and subsidence.

FIGS. 2A-2C are top, side and bottom views of an example of a spinal implant, such as spinal implant 102 of FIG. 1. Referring to FIG. 2A, a top view of spinal implant 202 is shown. In FIG. 2A, spinal implant 202 includes a plurality of keels 226 and defines a plurality of hollows 212. The keels 226 may include or correspond to anchoring elements 126 of FIG. 1A. As illustrated in FIG. 2A, two keels 226 protrude from a top portion of the body 112 of the spinal implant 202 and a single keel 226 protrudes from a bottom portion of the spinal implant 202.

Referring to FIG. 2B, a side view of the spinal implant 202 is shown. As illustrated in the example of FIG. 2B, the body 112 of the spinal implant 202 includes three injection tubes 222. The injection tubes 222 may include or correspond to the injection tubes 122 of FIG. 1. In other implementations, the spinal implant may have more or fewer injection tubes.

In some implementations, the body 112 may include one or more other apertures 242 The one or more apertures 242 may be configured to receive one or more fasteners or a second fillable material. As

Referring to FIG. 2C, a bottom view of the spinal implant 202 is shown. As illustrated in the example of FIG. 2C, a single keel 226 protrudes from a bottom portion of the spinal implant 202. In other implementations, the body 112 may include more or fewer keels 226 on the bottom portion. For example, the body 112 may include symmetrical keels 226 on the top and bottom.

As shown in FIGS. 2A-2C, the keels 226 have a beveled shape. To illustrate, a cross section of the keels 226 is not symmetrical with respect to a front to back reference plane of the spinal implant 202. The beveled shape (e.g., beveled edge) may help the spinal implant 202 to be inserted into position. For example, the spinal implant 202 may be inserted sideways into the spine.

Additionally, the body 112 of spinal implant may include other types of anchoring elements, such as stems. The stems may have a more spherical cross section and be smaller in diameter as compared to the keels of FIGS. 2A-2C.

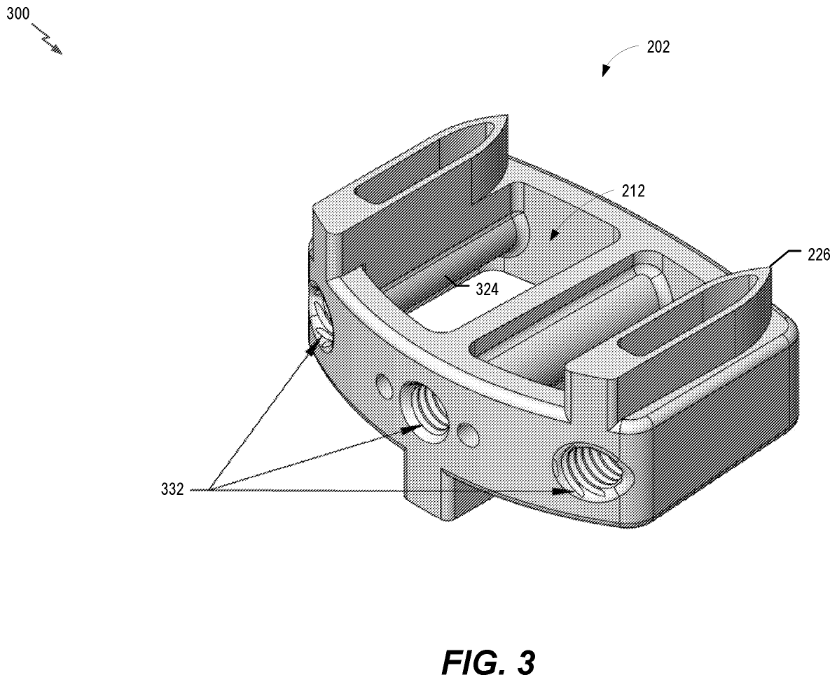

FIG. 3 is a perspective view 300 of the example of the spinal implant 202 of FIGS. 2A-2C. Perspective view 300 illustrates the spinal implant 202 from a top right perspective above the top side keels 226. In the example of FIG. 3, the injection tubes 222 include threads 332. Additionally, one or more channels 324 are illustrated which connect the injection tubes 222 to the keels 226.

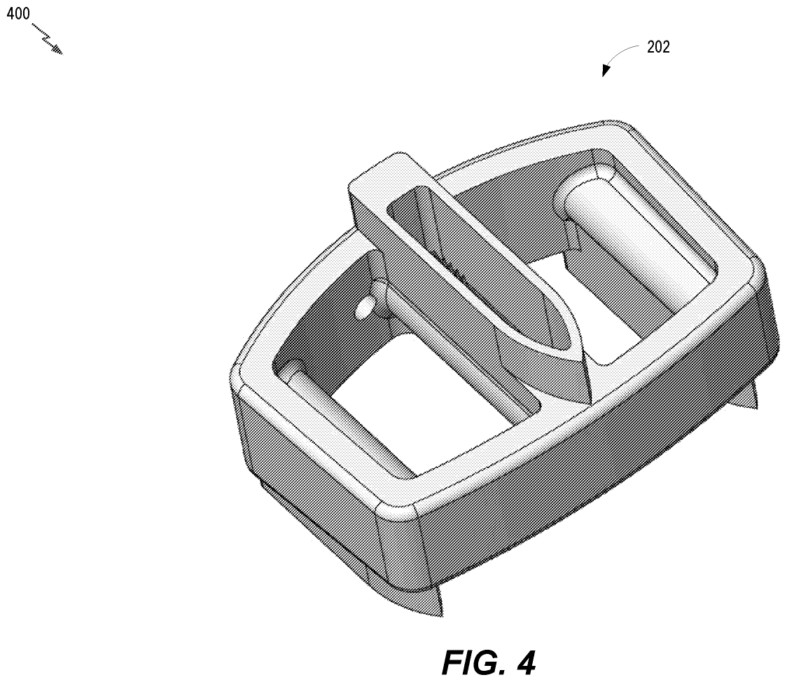

FIG. 4 is another perspective view 400 of the example of the spinal implant 202 of FIGS. 2A-2C. Perspective view 400 illustrates the spinal implant 202 from a bottom right perspective below the bottom side keel 226.

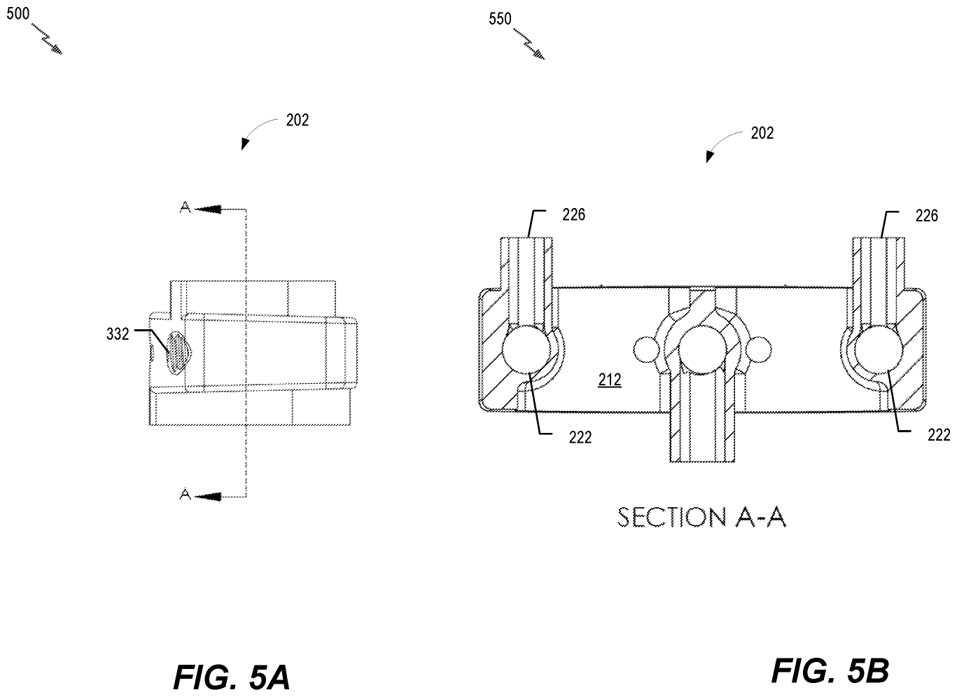

FIGS. 5A and 5B illustrate side and cross-section views of the spinal implant 202. FIG. 5A illustrates a side profile view and section line AA. Referring to FIG. 5A, profile view 500 depicts section line AA through a central or mid portion of the body 112.

Referring to FIG. 5B, cross-section view 550 depicts a cross-section of spinal implant 202 along section line AA of FIG. 5A. As illustrated in FIG. 5B, the interior channels 124 of body 112 are coupled to corresponding keels 226.

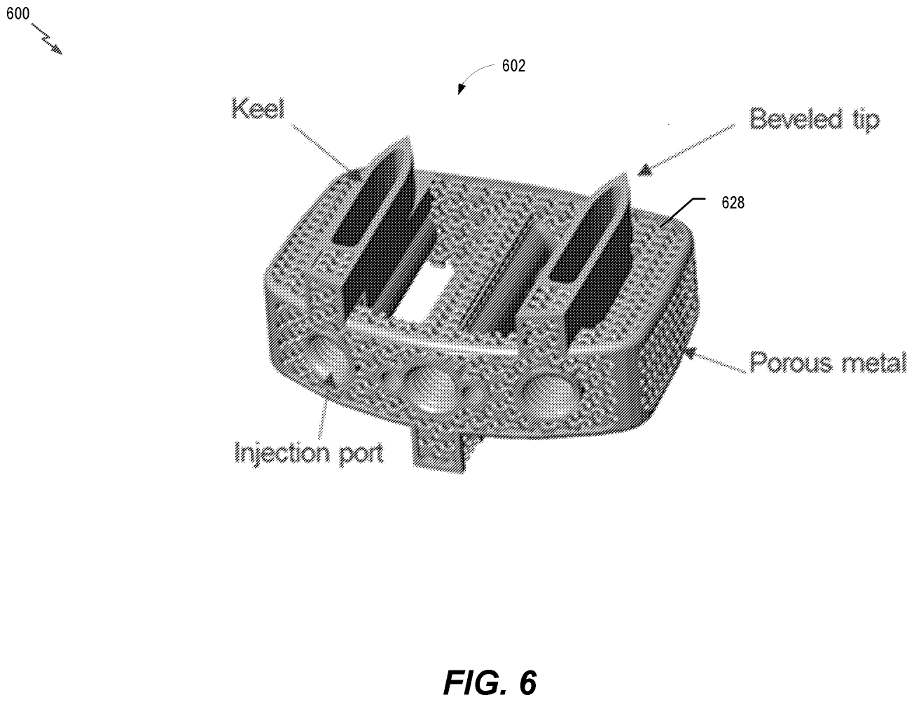

FIG. 6 illustrates an example of a spinal implant with a porous surface, such as the porous surface 132 of FIG. 1. As illustrated in the example of FIG. 6, the spinal implant 602 is similar to the spinal implant 202 and further includes a porous surface 628 on the exterior surfaces of body 112. In some implementations, the porous surface 628 is a porous metal surface, such as titanium or a titanium alloy.

FIG. 7 illustrates an exemplary cross-section view 700 of an example of a spinal implant 702 inserted into a spine 704. The exemplary spine 704 includes vertebrae 752-756 separated by disc space 772. The vertebrae 752-756 includes cancellous bone 762. As illustrated in the example of FIG. 7, a spinal implant 702 is inserted into a particular disc space 772 between two adjacent vertebrae of the spine 704. The spinal implant 702 is inserted into the disc space 772 and has keels which protrude into cancellous bone 762 of the two adjacent vertebrae 752 and 754. For example, the spinal implant 702 may be malleted or driven into place by force.

The spinal implant 702, once inserted, may receive injectable material 742, and may provide the injectable material 742 to the cancellous bone 762 of the two adjacent vertebrae 752 and 754.

Although the example of FIG. 7 illustrates a particular approach (e.g., anterior) into a particular area of the spine (e.g., lumbar), other the spinal implant and variants thereof may utilize different approaches and may be placed in other locations. For example, anterior, lateral, or posterior approaches may be used. As another example, cervical, lumbar, thoracic placement is possible. To illustrate, anterior approaches may include cervical and lumbar (acdf and alif) approaches, lateral approaches may include lumbar and thoracic (xlif) approaches, and posterior approaches may include transforaminal (tlif) and direct posterior (plif) approaches.

Additionally, although the defect/fusion in the example of FIG. 7 is for one disc space/two vertebral bodies, in other implementations, the defect may be larger. In such cases where larger defects are present, cage type spinal implants may be used.

FIG. 8 illustrates another example of a spinal implant 802. Spinal implant 802 is an alternative design that could be used for minimally invasive approaches via direct lateral, transforaminal, or posterior approaches in the lumbar spine. This includes but is not limited to a deployable keel 826 and an injection port 822. Deployable keep 826 is a deployable anchoring element and is coupled to injection port 822. Deployable keel 826 is configured to be advanced into the bone and to deliver material, provided to injection port 822, into the bone. As illustrated in the example of FIG. 8, deployable keel 826 includes two apertures to provide the material. Additionally, deployable keel 826 includes a beveled tip to help the keel 826 drive into the bone to deliver the material. The deployable keel 826 may be deployed/advanced into the bone by actuation, such as turning of a screw or nut.

In some implementations, spinal implant 802 includes one or more surface features 828. As illustrated in the example of FIG. 8, the surface features 828 include a plurality of grooves. The surface features 828 may provide a surface for bone ingrowth, adhesion, or both.

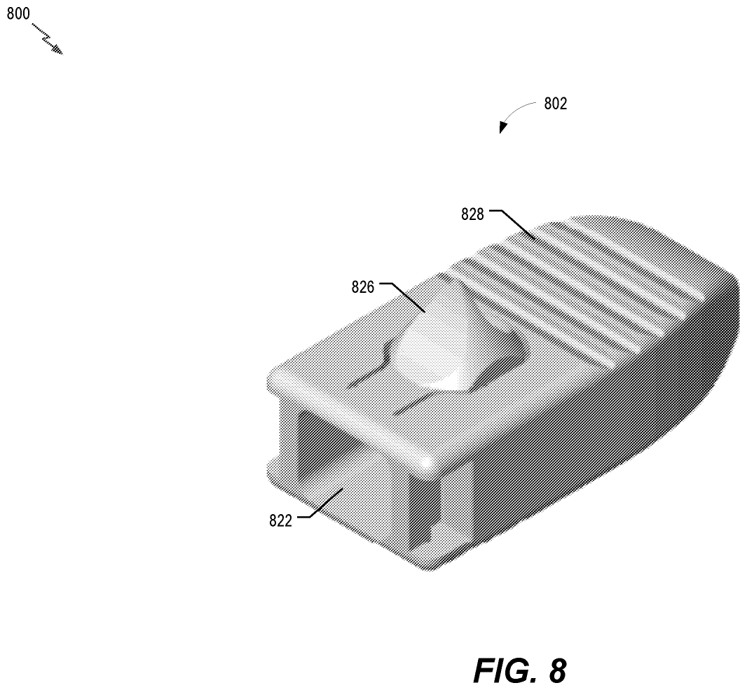

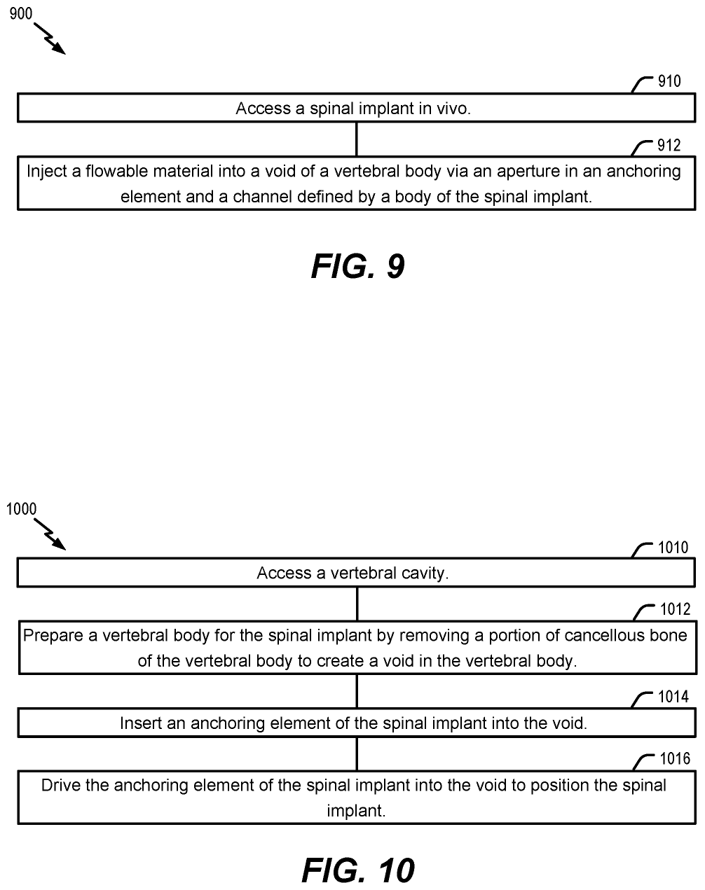

FIG. 9 illustrates a method 900 of using a spinal implant. The method 900 may be performed by a machine or a health care professional.

Method 900 includes accessing a spinal implant in vivo, at 910. Method 900 further includes injecting a flowable material into a void (e.g., prepared groove) of a vertebral body via an aperture in an anchoring element and a channel defined by a body of the spinal implant, at 912. Thus, method 900 describes operation of a spinal implant, and the spinal implant may enable increased fusion performance

FIG. 10 illustrates a method 1000 of using a spinal implant. The method 1000 may be performed by a machine or a health care professional. In some implementations, method 1000 is performed by one or more automated machines, which are controlled by one or more controllers.

Method 1000 includes accessing a vertebral cavity, at 1010. Method 1000 also includes preparing a vertebral body for the spinal implant by removing a portion of a vertebral endplate and a portion of cancellous bone of the vertebral body to create a void in the vertebral body, at 1012. For example, a groove may be drilled out of a vertebral endplate by a bone saw.

Method 1000 includes inserting an anchoring element of the spinal implant into the void, at 1014. Method 1000 further includes driving the anchoring element of the spinal implant into the void to position the spinal implant, at 1016. Thus, method 1000 describes operation of a spinal implant, and the spinal implant may enable increased fusion performance.

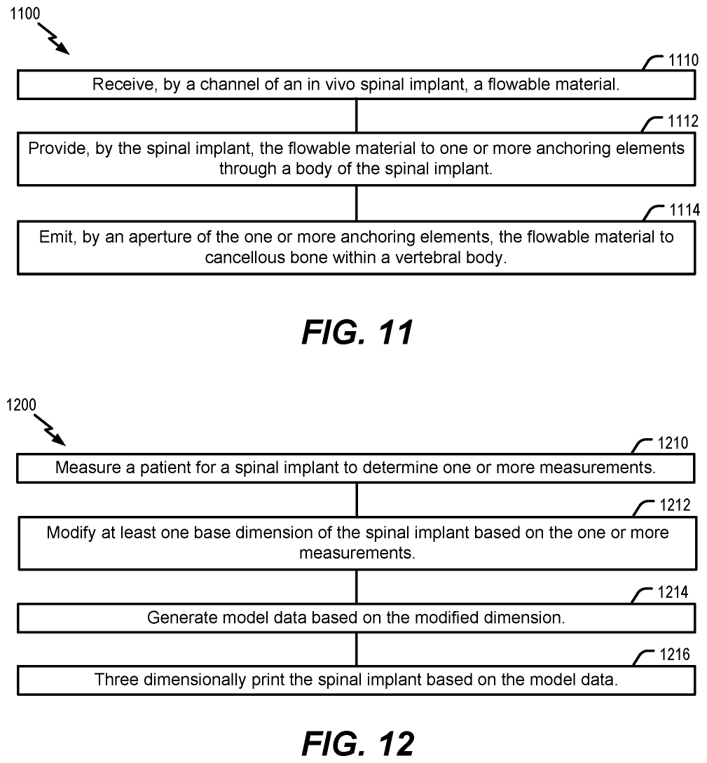

FIG. 11 illustrates a method 1100 of using a spinal implant. The method 1100 may be performed at or by a spinal implant, such as spinal implant 102.

Method 1100 includes receiving, by a channel of an in vivo spinal implant, a flowable material, at 1110. Method 1100 also includes providing, by the spinal implant, the flowable material to one or more anchoring elements through a body of the spinal implant, at 1112. Method 1100 further includes emitting, by an aperture of the one or more anchoring elements, the flowable material to a cancellous bone within a vertebral body, at 1114. Thus, method 1100 describes operation of a spinal implant, and the spinal implant may enable increased fusion performance.

FIG. 12 illustrates another method 1200 of manufacturing a spinal implant. The method 1200 may be performed at or by a computer, a 3D printer, or a combination thereof. Alternatively, the spinal implant may be manufacture with a tool and die, a mold, or another fabrication system, such as when fabricating custom or non-custom implants.

Method 1200 includes measuring a patient for a spinal implant to determine one or more measurements, at 1210. For example, a computer or health care professional may image a patient's spine to determine a measurement or may read a previously acquired image. To illustrate, an X-ray or CT image may be used to determine a spinal implant dimension, such as a vertebral spacing or thickness.

Method 1200 also includes modifying at least one base dimension of the spinal implant based on the one or more measurements, at 1212. To illustrate, for the example dimension of keel height, a base keel of X mm may be modified based on the vertebral spacing or thickness.

Method 1200 includes generating model data based on the modified dimension, at 1214. For example, a computer (e.g., a “slicing” program thereof) may generate 3D model data based on the modified keel height.

Method 1200 further includes three dimensionally printing the spinal implant based on the model data, at 1216. For example, a 3D printer may use the 3D model data to print the custom spinal implant, with the modified keel height.

It is noted that one or more operations described with reference to one of the methods of FIGS. 9-12 may be combined with one or more operations of another of FIGS. 9-12. For example, one or more operations of method 1000 may be combined with one or more operations of method 1100. Additionally, or alternatively, one or more operations described above with reference to FIG. 1 may be combined with one or more operations of FIG. 9, FIG. 10, FIG. 11, FIG. 12, or a combination of FIGS. 9-12.

### EXPERIMENTAL RESULTS

FIGS. 13-24 are directed to an experiment of implanting an example spinal implant with keels including delivery ducts into a cadaver spines. FIGS. 13-18 illustrate implantation procedures, including site preparation and delivery of the implant into the spine. FIGS. 19-24 illustrate spinal implants in vivo and after removal.

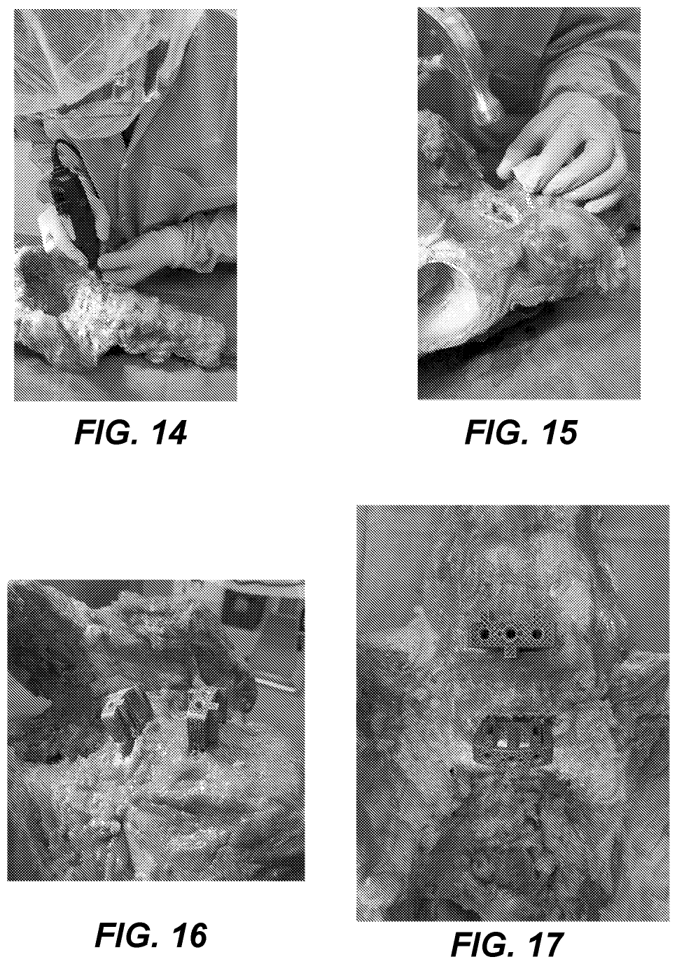

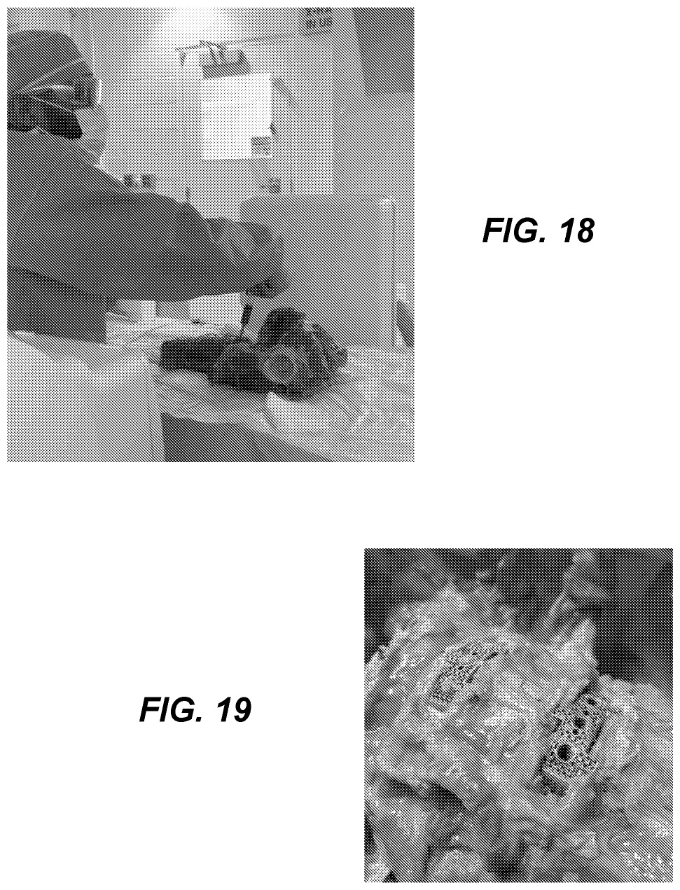

FIG. 13 illustrates an image of an injector device coupled to an injection port. FIG. 14 illustrates an image of bone site preparation. FIG. 15 illustrates an image of spinal implant insertion. FIG. 16 illustrates an image of spinal implants prior to insertion. FIG. 17 illustrates an image of in vivo spinal implants. FIG. 18 illustrates an image of in vivo injection of flowable materials to a spinal implant.

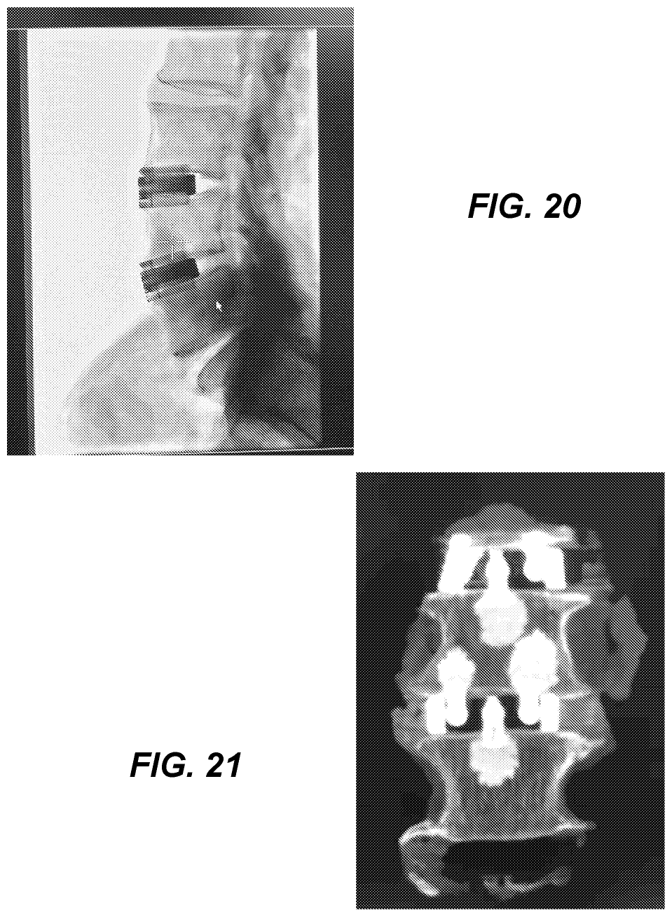

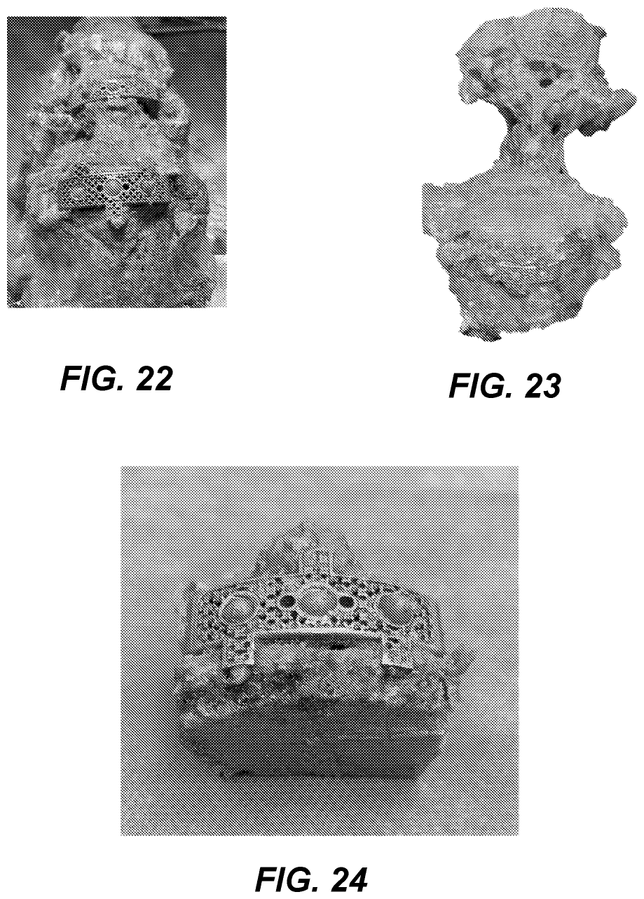

FIG. 19 illustrates an image of spinal implants after flowable material has been injected. FIG. 20 illustrates a CT image of spinal implants after insertion and injection. FIG. 21 illustrates an X-ray image of spinal implants after insertion and injection. FIG. 22 illustrates an image of periosteum and bone resected demonstrating cement in implant and bone. FIG. 23 illustrates an image of L3 transected demonstrating PMMA within L3 vertebral body. FIG. 24 illustrates an additional view after implant extraction.

In the experiment, three cadaveric lumbar spines with intact pelvises were used as a lumbar discectomy model. Two male and one female cadavers were used. Each cadaver was imaged using Computed tomography (CT) prior to interbody device implantation. One cadaver with a history of prostate cancer was found on CT imaging to have numerous blastic lesions throughout the spine. This cadaveric specimen was deemed not acceptable for PMMA injection and was used for device implantation and extraction testing only.

A novel 3D printed titanium interbody implant with anterior injection ports leading to open keels was fabricated, as shown in FIG. 7. Open keels were designed on both the superior and inferior surfaces. Two keels were fabricated on the superior surface and one keel on the inferior surface. The keels were fabricated with pointed tips to facilitate entry thru bone. PMMA: HV or MV dyed polymethylmethacrylate with gentamicin was used for injection into the spinal implants. A 10 ml syringe with nozzle adapter was used for injection of PMMA into the implant and vertebrae.

CT scans were obtained on each specimen prior to device implantation, after implantation prior to PMMA injection, and after PMMA injection. Fluoroscopy was used during PMMA injection to confirm the volume and location of the PMMA within the vertebral body.

Standard discectomy was performed at L4-5 on each of the cadavers. End plate preparation was performed to remove disc and cartilage to expose bone in the central portion of the vertebrae. Manual testing was performed in flexion and extension to assess the degree of motion and potential instability. The implant was then placed at the anterior aspect of the defect, with the keels resting on the anterior aspect of the vertebral bodies. The location of each keel was noted on the bone with a marker. A 1 mm drill was then used to create the entry channel in the bone for the keels. Bone channels, approximately 1 cm deep, 1 mm in height were made. The implant was then positioned, aligning the keels to the bone channels. The implant was then malleted in place, such that the anterior surface of the implant rested flush with the anterior lip of the vertebral bodies. The implant was then assessed via visual inspection to determine whether any gapping was noted between the implant and the vertebral bodies.

Manual testing in flexion and extension was performed to assess implant stability. The spines were then imaged. One spine was then selected to perform an additional, adjacent level (L3-4) discectomy and device implantation. A standard discectomy and end plate preparation was performed at L3-4. PMMA was then mixed and injected thru the anterior ports in the interbody implant using a 10 ml syringe. The PMMA was injected under fluoroscopic guidance. Approximately 1-3 ml of PMMA was injected thru each port. Once the PMMA cured, the spines were taken thru manual flexion/extension testing.

Interbody device implantation was successful in all four implants without endplate or vertebral body fracture. Integration of keel into vertebral body without gapping occurred in eight out of nine keel-vertebral body interfaces. Gapping (i.e., a space of greater than 1 mm) occurred in a single keel-vertebral body interface.

Three of the four implants were injected with PMMA. One of the implants was excluded from PMMA injection due to blastic lesions within the vertebral bodies secondary to metastatic prostate cancer. PMMA was successfully injected into all three of the remaining implants, completely filling the open keels in (i.e., all nine keels). PMMA entered the vertebral bodies via the keel-vertebral body interfaces, as shown in FIGS. 20 and 21. In eight of the nine keel-vertebral body interfaces the PMMA was contained within the appropriate potential space (i.e., vertebral bodies and keels) without extrusion. In a single keel-vertebral body interface (noted to have gapping after insertion), the PMMA extruded into the potential space within the interbody device. With this proposed design, this space is intended for graft material, and this space would not be empty or filled with the PMMA.

Stability was assessed in two ways: via motion between the implant-vertebral body interface and ease of implant extraction. All four implants were assessed for stability.

In all cases, the implants stabilized the discectomy defect even prior to PMMA injection. No implant could be translated out of the interbody space with flexion-extension thru the lumbar spines. Gapping however did occur in each of the implant-vertebral body interfaces at the extremes of flexion. All four implants could be extracted using extraction tools prior to injection of PMMA.

Three of the four implants were injected with PMMA. Two of the implants that were injected were placed at adjacent levels at L3-4 and L4-5 in the single male cadaver spine. The third implant was placed in the L4-5 interbody space in the female cadaver spine. After the PMMA cured, no gapping or translation was noted at any of the implant-vertebral body interfaces during flexion-extension testing. All three of the implants injected with PMMA were rigidly fixed and could not be extracted from the interbody space. All three implants were removed by performing osteotomies, as shown in FIGS. 22-24, in the bone surrounding the implants.

The above specification and examples provide a complete description of the structure and use of illustrative examples. Although certain aspects have been described above with a certain degree of particularity, or with reference to one or more individual examples, those skilled in the art could make numerous alterations to aspects of the present disclosure without departing from the scope of the present disclosure. As such, the various illustrative examples of the methods and systems are not intended to be limited to the particular forms disclosed. Rather, they include all modifications and alternatives falling within the scope of the claims, and implementations other than the ones shown may include some or all of the features of the depicted examples. For example, elements may be omitted or combined as a unitary structure, connections may be substituted, or both. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one example or may relate to several examples. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims. The claims are not intended to include, and should not be interpreted to include, means-plus- or step-plus-function limitations, unless such a limitation is explicitly recited in a given claim using the phrase(s) “means for” or “step for,” respectively.

1. A spinal implant comprising:
a body defining one or more injection ports and one or more channels, the one or more injection ports configured to receive flowable material and to provide the flowable material to the one or more channels; and
one or more anchoring elements protruding from a surface of the body, the one or more anchoring elements each defining an aperture coupled to the one or more channels and configured to receive the flowable material from the one or more channels and to provide the flowable material from the aperture.

2. The spinal implant of claim 1, wherein the one or more anchoring elements comprise one or more keels.

3. The spinal implant of any of claim 1 or 2, wherein at least one keel of the one or more keels comprises a beveled tip.

4. The spinal implant of any of claims 1-3, wherein the one or more channels include a plurality of channels, and wherein the one or more anchoring elements include a plurality of keels, wherein a corresponding aperture of each keel is coupled to a corresponding channel.

5. The spinal implant of any of claims 1-4, wherein the spinal implant comprises a vertebral implant or an intervertebral implant.

6. The spinal implant of any of claims 1-5, wherein the flowable material comprises polymethylmethacrylate (PMMA).

7. The spinal implant of any of claims 1-6, wherein the flowable material includes cells, bone cement, grafts, antibiotics, bisphosphonates, denosamab, or a combination thereof.

8. The spinal implant of any of claims 1-7, wherein the one or more anchoring elements are on a same side of the body.

9. The spinal implant of any of claims 1-8, wherein the body further comprises one or more second anchoring elements on a second side of the body opposite the side of the body.

10. The spinal implant of claim 9, wherein a number of the second anchoring elements is larger than a number of the anchoring elements.

11. The spinal implant of any of claims 1-10, wherein the one or more anchoring elements comprise one or more stems configured to deliver the flowable material to cancellous bone of a vertebral body of a patient.

12. The spinal implant of any of claims 1-11, wherein at least a portion of the surface of the body is porous.

13. The spinal implant of any of claims 1-12, wherein the body comprises a spinal cage implant and defines two hollow interior sections.

14. The spinal implant of any of claims 1-13, wherein the body comprises titanium.

15. The spinal implant of any of claims 1-13, wherein the body comprises polyetheretherketone (PEEK), carbon fiber, titanium, or a combination thereof.

16. A method of using a spinal implant, the method comprising:
accessing a spinal implant in vivo; and
injecting a flowable material into a void of a vertebral body via an aperture in an anchoring element and a channel defined by a body of the spinal implant.

17. The method of claim 16, further comprising injecting a second flowable material into the void of the vertebral body via the aperture in the anchoring element and the channel.

18. The method of any of claim 16 or 17, further comprising injecting the flowable material into a second void of a second vertebral body via a second aperture in a second anchoring element and a second channel defined by the body of the spinal implant.

19. A method of using a spinal implant, the method comprising:
accessing a vertebral cavity;
preparing a vertebral body for the spinal implant by removing a portion of a vertebral endplate and a portion of cancellous bone of the vertebral body to create a void in the vertebral body;
inserting an anchoring element of the spinal implant into the void; and
driving the anchoring element of the spinal implant into the void to position the spinal implant.

20. The method of claim 19, wherein removing the portion of the cancellous bone includes creating a groove in the vertebral body with a surgical saw or drill.

21. The method of any of claim 19 or 20, wherein inserting the anchoring element of the spinal implant into the void includes:
placing the implant in the proximate to the void; and
tapping the implant to drive the anchoring element into the void.

22. The method of any of claims 19-21, wherein inserting the anchoring element of the spinal implant into the void includes inserting a screw into the spinal implant, wherein insertion of the screw causes the spinal implant to expand and drive the anchoring element into the void.

23. A method of using a spinal implant, the method comprising:
receiving, by a channel of an in vivo spinal implant, a flowable material;
providing, by the spinal implant, the flowable material to one or more anchoring elements through a body of the spinal implant; and
emitting, by an aperture of the one or more anchoring elements, the flowable material to a cancellous bone within a vertebral body.

24. The method of claim 23, wherein the flowable material is curable, and further comprising curing the flowable material.

25. The method of any of claim 23 or 24, further comprising emitting, by a second aperture of the one or more anchoring elements, the flowable material to cancellous bone within a second vertebral body.

26. The method of any of claims 23-25, further comprising emitting, by a third aperture of the one or more anchoring elements, the flowable material to a vertebral space.

27. The method of any of claims 23-26, further comprising emitting, by the aperture of the one or more anchoring elements, second flowable material to the vertebral body.

28. The method of any of claims 23-27, wherein the flowable material comprises PMMA.

29. The method of any of claim 27 or 28, wherein the second flowable material comprises bone graft material.

30. A kit for a spinal implant, the kit comprising:
the spinal implant of claim 1; and
flowable material.

31. The kit of claim 30, wherein the flowable material includes PMMA.

32. The kit of any of claim 30 or 31, wherein the flowable material includes cells, bone cement, grafts, and/or other biological materials.

33. The kit of any of claims 30-32, further comprising a drill, a saw, a scraper, or other bone preparation equipment.

34. The kit of any of claims 30-33, further comprising spine access equipment.

35. The kit of any of claims 30-34, further comprising sterilization equipment.

36. The kit of any of claims 30-35, further comprising a syringe or another injecting device.

37. A method of manufacturing a spinal implant:
measuring a patient for a spinal implant to determine one or more measurements;
modifying at least one base dimension of the spinal implant to generate a modified dimension based on the one or more measurements;
generating model data based on the modified dimension; and
three dimensionally printing the spinal implant based on the model data.

38. A spinal implant comprising:
a body defining one or more injection ports and one or more channels, the one or more injection ports configured to receive flowable material and to provide the flowable material to the one or more channels; and
one or more anchoring elements protruding from a surface of the body, the one or more anchoring elements each defining an aperture coupled to the one or more channels and configured to receive the flowable material from the one or more channels and to provide the flowable material from the aperture.

39. The spinal implant of claim 38, wherein the one or more anchoring elements comprise one or more keels.

40. The spinal implant of claim 39, wherein at least one keel of the one or more keels comprises a beveled tip.

41. The spinal implant of claim 38, wherein the one or more channels include a plurality of channels, and wherein the one or more anchoring elements include a plurality of keels, wherein a corresponding aperture of each keel is coupled to a corresponding channel.

42. The spinal implant of claim 38, wherein the spinal implant comprises a vertebral implant or an intervertebral implant.

43. The spinal implant of claim 38, wherein the flowable material comprises polymethylmethacrylate (PMMA).

44. The spinal implant of claim 38, wherein the flowable material includes cells, bone cement, grafts, antibiotics, bisphosphonates, denosamab, or a combination thereof.

45. The spinal implant of claim 38, wherein the one or more anchoring elements are on a same side of the body.

46. The spinal implant of claim 38, wherein the body further comprises one or more second anchoring elements on a second side of the body opposite the side of the body.

47. The spinal implant of claim 46, wherein a number of the second anchoring elements is larger than a number of the anchoring elements.

48. The spinal implant of claim 38, wherein the one or more anchoring elements comprise one or more stems configured to deliver the flowable material to cancellous bone of a vertebral body of a patient.

49. The spinal implant of claim 38, wherein at least a portion of the surface of the body is porous.

50. The spinal implant of claim 38, wherein the body comprises a spinal cage implant and defines two hollow interior sections.

51. The spinal implant of claim 38, wherein the body comprises titanium.

52. The spinal implant of claim 38, wherein the body comprises polyetheretherketone (PEEK), carbon fiber, titanium, or a combination thereof.

53. A method of using a spinal implant, the method comprising:
accessing a spinal implant in vivo; and
injecting a flowable material into a void of a vertebral body via an aperture in an anchoring element and a channel defined by a body of the spinal implant.

54. The method of claim 53, injecting a second flowable material into the void of the vertebral body via the aperture in the anchoring element and the channel.

55. The method of claim 53, injecting the flowable material into a second void of a second vertebral body via a second aperture in a second anchoring element and a second channel defined by the body of the spinal implant.

56. A method of using a spinal implant, the method comprising:
accessing a vertebral cavity;
preparing a vertebral body for the spinal implant by removing a portion of a vertebral endplate and a portion of cancellous bone of the vertebral body to create a void in the vertebral body;
inserting an anchoring element of the spinal implant into the void; and
driving the anchoring element of the spinal implant into the void to position the spinal implant.

57. The method of claim 56, wherein removing the portion of the cancellous bone includes creating a groove in the vertebral body with a surgical saw or drill.

58. The method of claim 56, wherein inserting the anchoring element of the spinal implant into the void includes:
placing the implant in the proximate to the void; and
tapping the implant to drive the anchoring element into the void.

59. The method of claim 56, wherein inserting the anchoring element of the spinal implant into the void includes inserting a screw into the spinal implant, wherein insertion of the screw causes the spinal implant to expand and drive the anchoring element into the void.

60. A method of using a spinal implant, the method comprising:
receiving, by a channel of an in vivo spinal implant, a flowable material;
providing, by the spinal implant, the flowable material to one or more anchoring elements through a body of the spinal implant; and
emitting, by an aperture of the one or more anchoring elements, the flowable material to a cancellous bone within a vertebral body.

61. The method of claim 60, wherein the flowable material is curable, and further comprising curing the flowable material.

62. The method of claim 60, further comprising emitting, by a second aperture of the one or more anchoring elements, the flowable material to cancellous bone within a second vertebral body.

63. The method of claim 60, further comprising emitting, by a third aperture of the one or more anchoring elements, the flowable material to a vertebral space.

64. The method of claim 60, further comprising emitting, by the aperture of the one or more anchoring elements, second flowable material to the vertebral body.

65. The method of claim 64, wherein the flowable material comprises PMMA.

66. The method of claim 64, wherein the second flowable material comprises bone graft material.

67. A kit for a spinal implant, the kit comprising:
the spinal implant of claim 1; and
flowable material.

68. The kit of claim 67, wherein the flowable material includes PMMA.

69. The kit of claim 67, wherein the flowable material includes cells, bone cement, grafts, and/or other biological materials.

70. The kit of claim 67, further comprising a drill, a saw, a scraper, or other bone preparation equipment.

71. The kit of claim 67, further comprising spine access equipment.

72. The kit of claim 67, further comprising sterilization equipment.

73. The kit of claim 67, further comprising a syringe or another injecting device.

74. A method of manufacturing a spinal implant:
measuring a patient for a spinal implant to determine one or more measurements;
modifying at least one base dimension of the spinal implant to generate a modified dimension based on the one or more measurements;
generating model data based on the modified dimension; and
three dimensionally printing the spinal implant based on the model data.
